(19) United States
(12) Patent Application Publication
(10) Pub. No.: US 2014/0276630 A1
(43) Pub. Date: Sep. 18, 2014

(54) SYRINGE WITH RETRACTOR

(71) Applicant: Aurora Pharmaceutical, LLC, Northfield, MN (US)

(72) Inventors: James L. Pokorney, Northfield, MN (US); Mark G. Werner, Northfield, MN (US); Michael A. Strobel, Northfield, MN (US)

(73) Assignee: Aurora Pharmaceutical, LLC, Northfield, MN (US)

(21) Appl. No.: 14/218,306

(22) Filed: Mar. 18, 2014

Related U.S. Application Data
(60) Provisional application No. 61/792,056, filed on Mar. 15, 2013.

(51) Int. Cl. A61D 7/00; A61M 5/315

(52) U.S. Cl. CPC A61D 7/00; A61M 5/31513; A61M 2202/0007
USPC 604/514; 604/73

(57) ABSTRACT

This invention encompasses products and methods for preventing teat infections in cows resulting from a variety of causes. More specifically, this invention relates to products and methods for blocking a cow's teat and teat canal to prevent infection during a non-lactating period.

### FIELD OF THE INVENTION

This invention relates to an improved product and method for preventing infections in cows resulting from a variety of causes. More specifically, this invention relates to a method for blocking a cow's teat and teat canal to prevent infection during a non-lactating period.

### BACKGROUND OF THE INVENTION

Mastitis is a common disease of dairy cattle. Mastitis is an inflammatory response of the udder tissue due to some form of injury, the most common being bacterial infection. The inflammatory response consists of an increase in blood proteins and white blood cells in the mammary tissue and the milk.

To prevent new cases of mastitis farmers often treat cows with a prophylactic treatment of an intramammary antibiotic. This is administered in the form of a paste or gel infused with antibiotic. A syringe is used to insert the material directly into the teat canal via the opening at the base of the teat.

### BRIEF DESCRIPTION OF THE FIGURES

FIG. 1 shows possible prior art.

FIG. 2 shows a preferred embodiment using a standard syringe and a newly designed retractor. The retractor can be “snapped” into place. A prototype similar to this retractor design was fabricated using SLA process and snapped over a standard 1 ml BD syringe.

FIG. 3 shows a preferred method of operation in the cow teat application. The syringe tip is inserted into the teat canal about 3 mm. A sealant liquid is injected into the teat. When the leading distal edge of the retractor encounters the teat surface, the piston stroke is not yet completed. To complete the stroke, the syringe barrel is required to retract proximally, thereby retracting the syringe barrel tip out of the teat canal. During this retraction process, the final portion of sealant liquid is expelled to fill the now evacuated teat canal. To those knowledgeable in the art, it would be apparent that the particular length of the retractor can be adjusted to effect the specific distance of plunger retraction relative to plunger stroke travel. Tip retraction distance could range from zero to total plunger travel distance.

FIG. 4 shows another preferred embodiment.

FIG. 5 shows a schematic of operation.

### SUMMARY OF THE INVENTION

The present invention is directed to a method of blocking a cow's teat and teat canal to prevent infection during a non-lactating period.

One embodiment of the present invention is directed to a method of treating the teat by providing a syringe that retracts away from the site of injection at end of plunger stroke to allow placement of material into the teat canal itself This allows for uniform placement of material in the teat canal as compared to currently marketed syringes which rely on the operator's unpredictable application.

Specifically, the invention has the following advantage: To inject a fluid contained within a syringe into a body, the end of the syringe (blunt or needle) needs to be inserted, at least partially, into the body. This new invention directs the inserted portion of the syringe to be retracted away from the body at the end of the plunger stroke to allow expelled fluid to fill the body area previously occupied by the inserted end of the syringe (blunt or needle). This new approach helps to prevent infection and may also reduce the incidence of Mastitis which is most often transmitted by contact with the milking machine, and through contaminated hands or materials.

### DETAILED DESCRIPTION OF THE INVENTION

In a preferred embodiment, the new invention can be used to inject a sealant into a cow teat. In this embodiment, the syringe tip is generally short and blunt. In other embodiments, the syringe tip could be long and sharp (i.e., needle). The invention principle works in either case. For convenience, a cow teat embodiment will be described, but for those knowledgeable in the art other applications using the retractor principle are also feasible.

In another preferred embodiment, this invention helps to ensure the teat skin is in good condition, maintain skin moisture and natural elasticity and helps the teat to restrict the development of hyperkeratosis. Using a high quality teat disinfectant (or even antibiotic in some cases), carefully applied, helps to reduce hyperkeratosis.

Elements

Syringe Barrel

A standard syringe barrel is employed with an open tip on distal end and finger grips near proximal end.

Syringe Plunger

A typical syringe plunger is employed that fits within the barrel with a piston on one end and a thumb surface on the other.

Syringe Retractor

Compared to prior art syringes containing only barrel and plunger, in new invention a syringe retractor is used. The retractor is connected to the syringes plunger at its proximal end. The retractor is generally coaxial with the syringe barrel and larger in diameter. When the user advances the plunger, the retractor advances as well. It is adjacent but slideable with regard to the syringe barrel. The plunger and retractor could be manufactured as one assembly and, as is apparent to one of ordinary skill in the art, the plunger and retractor can be made of biodegradable plastic for ease of use or alternatively one use before being disposed.

As illustrated in FIG. 5, the three elements (Barrel, Plunger, and Retractor) and the target Surface are shown schematically.

1. The end of the Barrel is inserted into the Surface

2. The Plunger and Retractor are advanced relative to the Barrel until the Retractor makes contact with Surface. During this movement, the Plunger and Retractor generally advance towards the Surface while the Barrel remains generally stationary relative to the Surface.

3. When Retractor advancement is stopped by the Surface, the Plunger can be further advanced relative to Barrel by retracting the Barrel away from the Surface.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

We claim:

1. A hand-held fluid delivery apparatus, comprising (a) a syringe barrel having a fluid receptacle; (b) a plunger disposable within the fluid receptacle of the syringe barrel and moveable along a length of the barrel along a longitudinal axis; and (c) a retractor connected to the plunger at its proximal end, for injecting a liquid into a target surface through the end of the syringe barrel, whereby the plunger can be further advanced relative to the barrel by retracting the barrel away from the surface.

2. A hand-held fluid delivery apparatus of claim 1, wherein the fluid is a sealant.

3. A hand-held fluid delivery apparatus of claim 2, wherein the fluid also contains a high quality teat disinfectant.

4. A hand-held fluid delivery apparatus of claim 2, wherein the fluid also contains an antibiotic.

5. A hand-held delivery apparatus of claim 1, wherein the syringe barrel comprises a syringe tip, and wherein the hand-held syringe further comprises one of a catheter and a needle connected to the syringe tip to accept a fluid discharge from the syringe barrel as the plunger is advanced through the fluid receptacle.

6. A hand-held delivery apparatus of claim 5, wherein the syringe tip is short and blunt.

7. A hand-held delivery apparatus of claim 5, wherein the syringe tip is long and sharp.

8. A hand-held fluid delivery apparatus of claim 1, wherein the target surface is a teat.

9. A hand-held fluid delivery apparatus, comprising (a) a syringe barrel having a fluid receptacle; (b) a plunger disposable within the fluid receptacle of the syringe barrel and moveable along a length of the barrel along a longitudinal axis; and (c) a retractor connected to the plunger at its proximal end, for injecting a liquid into a target surface through the end of the syringe barrel, whereby the leading distal edge of the retractor encounters the teat surface, the syringe barrel is required to retract proximally, thereby retracting the syringe barrel tip out of the teat canal and filling the now evacuated teat canal.

10. A method for reducing the incidence of mastitis in an animal by introducing a composition into the teat canal of the animal comprising the steps of:
(a) inserting the tip of a syringe into the teat canal;
(b) injecting a sealant liquid into the teat;
(c) retracting the syringe barrel tip out of the teat canal; and
(d) expelling the final portion of sealant liquid to fill the now evacuated teat canal.

11. The method according of claim 10, wherein the method is employed during the non-lactating period of the animal.

12. The method according to claim 10, wherein the animal is a cow

13. The method according to claim 10, wherein the composition is a paste.

14. The method according to claim 10, wherein the composition is a gel.

15. The method according to claim 10, wherein the composition comprises keratin.
